(19) United States
(12) Patent Application Publication
(10) Pub. No.: US 2002/0034811 A1
(43) Pub. Date: Mar. 21, 2002

(54) METHOD FOR REMOVAL OF PROTEASE ACTIVITY FROM AMYLASE

(76) Inventor: ANNE G. ROWLANDS, HONEOYE FALLS, NY (US)

Correspondence Address: PATENT LEGAL STAFF, EASTMAN KODAK COMPANY, 343 STATE STREET, ROCHESTER, NY, 14650-2201, US

(21) Appl. No.: 09/182,936

(22) Filed: Oct. 30, 1998

(51) Int. Cl. C12N 9/26

(52) U.S. Cl. 435/201; 435/184

(57) ABSTRACT

The present invention is a process for removal of protease activity from heat stable amylases. A heat stable amylase solution is provided and heated from 70 to 100° C. for a time sufficient to denature protease activity. The heat stable amylase solution is subsequently filtered.

### CROSS REFERENCE TO RELATED APPLICATIONS

This application relates to commonly assigned copending application Ser. No. ______, (Docket 78,452) filed simultaneously herewith incorporated by reference herein. This application relates to commonly assigned copending application Serial No. , (Docket 78,454), filed simultaneously herewith incorporated by reference herein.

### FIELD OF THE INVENTION

The present invention relates to amylase solutions and more particularly to a method for reducing protease activity in amylase solutions.

### BACKGROUND OF THE INVENTION

Amylases are a group of widely occurring hydrolases which cleave the alpha 1,4-glyosidic bonds in oligosaccharides and polysaccharides like starch, glycogen and dextrans. Commercially available amylases produced through fermentation technology typically also contain contaminating protease activity. In applications such as the starch industry or the laundry detergent industry the presence of contaminating protease activity in an amylase solution is of no concern or an advantage. Application of amylases in cases in which the integrity of a protein is necessary require that amylase be free of protease contamination. The application of amylase for the removal of gelatin insolubles in a gelatin solution requires amylase free of protease U.S. Ser. No. ______ (Docket 78,452) Methods of removing contaminating proteases from amylase by ion exchange resins and gamma ray irradiation have been described in U.S. Pat. Nos. 3416,997 and 3,642,582 and Japanese Abstract 51/76481.

An object of the present invention is to provide a rapid cost effective method for the removal of protease activity from heat stable amylases.

### SUMMARY OF THE INVENTION

The present invention is a process for removal of protease activity from heat stable amylases. A heat stable amylase is provided and heated to 70-100° C. for a time sufficient to denature contaminating proteases followed by filtration.

### DETAILED DESCRIPTION OF THE INVENTION

The present invention is a process for removal of protease activity from heat stable amylases. A heat stable amylase solution is provided and heated from 70 to 100° C. for a time sufficient to denature protease activity. The heat stable amylase solution is subsequently filtered.

Amylases

| | |
|---|---|
| Maxamyl L | Genencor International |
| Termamyl 12L Type L | Novo Nordisk |

Amylase Activity:

Amylase activity was monitored using the amylase activity slide kit for the Ektachem DT60 Analyzer. This assay is based on the hydrolysis of dyed starch to smaller dyed saccharides. Intensity of color generation is proportional to amylase activity.

Protease Activity:

Protease activity was monitored using a gelatin substrate. Briefly, gelatin was incubated with amylase solutions for varying times at 50° C. The molecular weight distribution of photographic gelatin was monitored to detect protease activity. Protease activity was evident by shift in the molecular weight distribution profile of gelatin toward smaller peptides.

Molecular weight distribution of gelatin was determined by high- performance liquid chromatography in the aqueous size exclusion mode. Gelatin samples were dissolved in the chromatographic eluent, a phosphate buffer containing sodium dodecyl sulfate, and separated on a Toso Haas TSK Gel size exclusion column. The effluent was monitored with a UV detector set at 220 nm. Known molecular weight standards were used to prepare a calibration curve, which was constructed by plotting the log of molecular weight versus retention time. The molecular weight distribution of gelatin samples was determined from the linear portion of this calibration curve.

Due to variable bond breakage during manufacture, gelatin is composed of a distribution of proteins of varying lengths. Aqueous size exclusion chromotagraphy provides a method of analysis for determining the gelatin molecular weight distribution. This distribution is described as containing the following five fractions; high molecular weight or HMW (>250 daltons); Beta (250-150 Kdaltons); Alpha (150-50 Kdaltons); Subalpha (50-20 Kdaltons); and low molecular weight or LMW (20-4 Kdaltons). In general, high gel strength correlates with high gelatin alpha fraction content, and high viscosity correlates with high gelatin HMW fraction content.

### EXAMPLE 1

Removal of Protease activity from Amylase Solutions.

Removal of contaminating proteases from amylase solutions was achieved by heating a 5% w/w solution of amylase in water to 85° C. for 1 hour followed by filtration. Diatomaceous earth was used as a filter aid. Protease activity was monitored by analyzing the molecular weight distribution of gelatin in the presence of the amylase following an incubation at 50° C. for 45 minutes. Gelatin molecular weight distribution was measured using aqueous size exclusion chromatography.

| | RELATIVE % AREA | | | | |
|---|---|---|---|---|---|
| | | BETA | ALPHA | SUB- | |
| | HMW | 250- | 150- | ALPHA | LMW |
| | >250 K | 150 K | 50 K | 50-20 K | <20 K |
| UNTREATED | | | | | |
| AMYLASES | | | | | |
| SOLUTIONS | | | | | |
| CONTROL GEL | 22.13 | 19.25 | 47.57 | 7.79 | 3.26 |
| CONTROL GEL + | 0 | 0 | 0 | 9.53 | 72.88 |
| 5% MAXAMYL | | | | | |
| CONTROL GEL + | 0 | 0 | 1.81 | 21.3 | 61.4 |
| 5% TERMAMYL | | | | | |
| HEAT & FILTER | | | | | |
| TREATED | | | | | |
| AMYLASE | | | | | |
| SOLUTIONS | | | | | |
| CONTROL GEL | 21.76 | 20.16 | 47.39 | 7.41 | 3.29 |
| CONTROL GEL + | 21.07 | 19.71 | 47.33 | 7.95 | 3.94 |
| 5% MAXAMYL | | | | | |
| CONTROL GEL + | 20.34 | 19.36 | 46.63 | 7.86 | 5.82 |
| 5% TERMAMYL | | | | | |

In the above table the control gelatin represents the molecular weight distribution of gelatin dissolved in water. The addition of commercially available Maxamyl or Termamyl (labeled as untreated amylases) to a gelatin solution results in rapid loss of high molecular weight gelatin and production of low molecular weight gelatin peptides due to contaminating protease activity. Following treatment of Maxamyl and Termamyl with heat and filtration, gelatin molecular weight distribution is maintained in the presence of treated amylase solutions, indicating that the treatment procedure has removed the protease activity in the amylase solutions.

### EXAMPLE 2

5% w/w amylase solutions were treated at varying temperatures for 1 hour followed by filtration. Diatomaceous earth was used as a filter aid. Optimization of conditions for removal of protease activity and minimization of amylase activity loss is enzyme dependent. Amylase activity in a 5% solution before and after protease removal is shown below. Amylase activity was measured using an Ektachem DT-60 amylase assay as previously described.

| | | Amylase Activity (5% solution) | |
|---|---|---|---|
| | Treatment | Before | After |
| | Temperature | Protease Removal | Protease Removal |
| Maxamyl | 85° C. | 382500 U/L | 315 U/L |
| | 80° C. | 431500 U/L | 33150 U/L |
| Termamyl | 85° C. | 153000 U/L | 102000 U/L |

Optimum conditions for removal of protease activity will vary for each amylase and is a function of the temperature stability of the amylase. As shown above Maxamyl is less heat stable than Termamyl. Retention of amylase activity for Maxamyl is improved by treatment at 80° C. versus 85° C. In contrast, Termamyl maintains much higher amylase activity than Maxamyl following heat treatment at 85° C. Control factors of time and temperature can be optimized for removal of protease activity and preservation of amylase activity.

The invention has been described in detail with particular reference to certain preferred embodiments thereof, but it will be understood that variations and modifications can be effected within the spirit and scope of the invention.

What is claimed is:

1. A process for purifying amylase solutions comprising:
providing a heat stable amylase solution;
heating the heat stable amylase solution from 70 to 100° C. for a time sufficient to denature protease activity; and
filtering the heat stable amylase solution.

2. The process of claim 1 wherein the time is at least 1 hour.

3. The process of claim 1 wherein the heat stable amylase solution is heated to from 80° C. to 85° C.
